(19) United States
(12) Patent Application Publication
(10) Pub. No.: US 2009/0035188 A1
(43) Pub. Date: Feb. 5, 2009

(54) FRAGRANCE EMITTING APPARATUS

(75) Inventors: XIAO-HONG HUANG, Shenzhen (CN); XIAO-LIN GAN, Shenzhen (CN); YU-KUANG HO, Tu-Cheng (TW)

Correspondence Address: PCE INDUSTRY, INC.;ATT. CHENG-JU CHIANG, 458 E. LAMBERT ROAD, FULLERTON, CA, 92835, US

(73) Assignees: HONG FU JIN PRECISION INDUSTRY (ShenZhen) CO., LTD., Shenzhen City (CN); HON HAI PRECISION INDUSTRY CO., LTD., Tu-Cheng (TW)

(21) Appl. No.: 11/960,745

(22) Filed: Dec. 20, 2007

(30) Foreign Application Priority Data
Aug. 3, 2007 (CN) 200710201266.2

(51) Int. Cl. A61L 9/03; G05D 23/00

(52) U.S. Cl. 422/105; 422/125

(57) ABSTRACT

A fragrance emitting apparatus includes a case, a heat conducting board mounted in the case, a fragrance container disposed in the case and contacting the heat conducting board, a heater disposed in the case and contacting the heat conducting board, and a temperature control apparatus connected to the heater and configured to control the heater to heat the heat conducting board according to the temperature thereof.

### BACKGROUND

1. Field of the Invention

The present invention relates to fragrance emitting apparatuses, and particularly to a fragrance emitting apparatus which is used in electronic devices to emanate fragrance.

2. Description of related art

Nowadays, electronic devices such as computers are widely used in many fields. If users operate electronic devices for a long time, they may feel tired, thereby influencing their health and working efficiency. Medical experts have that certain fragrances can have a beneficial influence on productivity of people in the workplace. Moreover, pleasant fragrances can mask foul smells like that of scorching of high speed processors emanating from computers.

What is desired, therefore, is to provide a fragrance emitting apparatus which is arranged in the electronic devices to emit a beneficial fragrance.

### SUMMARY

An embodiment of a fragrance emitting apparatus includes a case, a heat conducting board mounted in the case, a fragrance container disposed in the case and contacting the heat conducting board, a heater disposed in the case and contacting the heat conducting board, and a temperature control apparatus connected to the heater and configured to control the heater to heat the heat conducting board according to the temperature thereof.

Other advantages and novel features of the present invention will become more apparent from the following detailed description of an embodiment when taken in conjunction with the accompanying drawings, in which:

### BRIEF DESCRIPTION OF THE DRAWINGS

FIG. 1 is a schematic view of a fragrance emitting apparatus in accordance with an embodiment of the present invention; and

FIG. 2 is a circuit block diagram of the fragrance emitting apparatus of FIG. 1.

### DETAILED DESCRIPTION

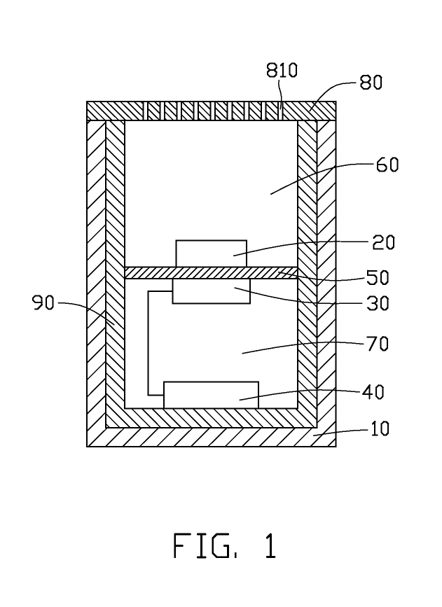

Referring to FIG. 1, a fragrance emitting apparatus in accordance with an embodiment of the present invention is configured in an electronic device to emit fragrance. The fragrance emitting apparatus includes a case 10, a fragrance container 20, a heater 30, a temperature control apparatus 40, and a heat conducting board 50.

The heat conducting board 50 is mounted in the case 10, and separates the case 10 into a first room 60 and a second room 70. The fragrance container 20 is disposed in the first room 60 and contacts the heat conducting board 50. The heater 30 and the temperature control apparatus 40 are disposed in the second room 70. The heater 30 contacts the heat conducting board 50. The fragrance container 20 contains a solid or liquid fragrance material, which can generate fragrance by activity of the heater 30. The heater 30 is configured to heat the fragrance container 20 via the heat conducting board 50. The temperature control apparatus 40 is configured to control temperature of the heater 30.

In this embodiment, the fragrance emitting apparatus further includes a cover 80 arranged on the case 10 and configured to allow exchange of the fragrance container 20. The cover 80 defines a plurality of through holes 810 configured for allowing fumes of the heated fragrance material contained in the fragrance container to escape the case 10. A heat insulation layer 90 is disposed in the case 10 and contacts the bottom and sidewall of the case 10.

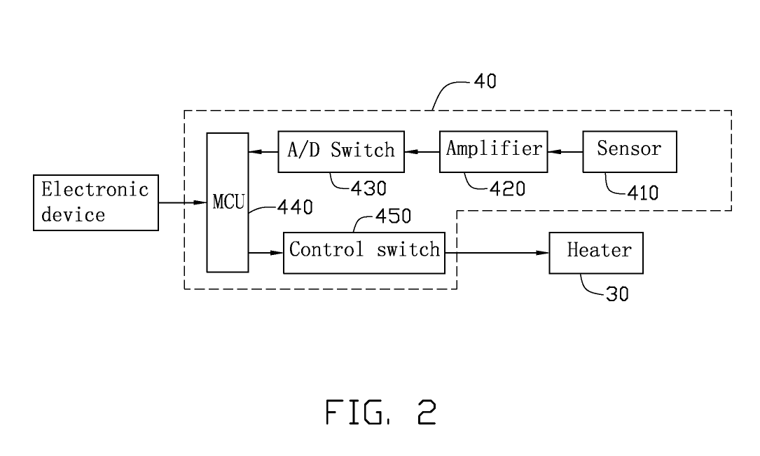

Referring also to FIG. 2, the temperature control apparatus 40 includes a sensor 410, an amplifier 420, an A/D switch 430, a micro control unit (MCU) 440, and a control switch 450. The sensor 410 contacts the heat conducting board 50 to sense the temperature of the heat conducting board 50. The sensor 410 is connected to a terminal of the MCU 440 via the amplifier 420 and the A/D switch 430 in turn. Another terminal of the MCU 440 is connected to the heater 30 via the control switch 450. A power terminal of the MCU 440 is connected to the electronic device to receive power therefrom.

In use, the sensor 410 senses the temperature of the heat conducting board 50 and sends corresponding temperature signals to the amplifier 420. The temperature signals are amplified by the amplifier 420 and changed to digital signals by the A/D switch, and then transmitted to the MCU 440. The MCU 440 controls the control switch 450 to control the heater to be turned on or off according to the temperature signals. When the temperature of the heat conducting board 50 is less then a predetermined temperature value, the MCU 440 controls the control switch 450 to control the heater to be turned on. When the temperature of the heat conducting board 50 is greater then the predetermined temperature value, the MCU 440 controls the control switch 450 to control the heater to be turned off.

In use, the fragrance emitting apparatus can be arranged in an appropriate place of the electronic device. The fragrance emitting apparatus can adjust emanating quantity of fumes from the fragrance container 20 according to need, which is very convenient.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A fragrance emitting apparatus comprising:
a case;
a heat conducting board mounted in the case;
a fragrance container containing a fragrance material disposed in the case and contacting the heat conducting board;
a heater disposed in the case and contacting the heat conducting board; and
a temperature control apparatus connected to the heater and configured to control the heater to heat the heat conducting board according to the temperature thereof.

2. The fragrance emitting apparatus as claimed in claim 1, wherein the temperature control apparatus comprises a sensor, an amplifier, an A/D switch, a micro control unit (MCU), and a control switch, the sensor senses the temperature of the heat conducting board and send corresponding temperature signals to the amplifier, the temperature signals is amplified by the amplifier and changed to digital signal by the A/D switch, and then transmitted to the MCU, the MCU controls the control switch to control the heater to be turned on or off according to the temperature signals.

3. The fragrance emitting apparatus as claimed in claim 1, further comprising a cover arranged on the case and configured to allow exchange of the fragrance bag.

4. The fragrance emitting apparatus as claimed in claim 3, wherein the cover defines a plurality of through holes configured to allow fumes of the heated fragrance material contained in the fragrance container to escape.

5. The fragrance emitting apparatus as claimed in claim 1, wherein a heat insulation layer is arranged in the case and contacts the bottom and sidewall.

6. The fragrance emitting apparatus as claimed in claim 1, wherein the fragrance material contained in the fragrance container is a solid or a liquid.

7. The fragrance emitting apparatus as claimed in claim 1, wherein the temperature control apparatus is disposed in the case.
